(19) United States
(12) Patent Application Publication
(10) Pub. No.: US 2024/0180612 A1
(43) Pub. Date: Jun. 6, 2024

(54) ACTIVE ELECTRODE FOR AN ELECTRIC SURGICAL INSTRUMENT

(71) Applicant: KARL STORZ SE & CO. KG, Tuttlingen (DE)

(72) Inventors: Martin HAHN, Tuttlingen (DE); Rainer HERMLE, Tuttlingen (DE); Laura SCHINDLER, Tuttlingen (DE)

(21) Appl. No.: 18/576,169

(22) PCT Filed: Jul. 6, 2022
(86) PCT No.: PCT/EP2022/068701

§ 371 Date: Jan. 3, 2024

(30) Foreign Application Priority Data
Jul. 7, 2021 (DE) 10 2021 117 566.6

(51) Int. Cl. A61B 18/14; A61B 18/00; A61B 18/12; A61B 18/16

(52) U.S. Cl. CPC A61B 18/149; A61B 18/1206; A61B 2018/00625; A61B 2018/1405; A61B 2018/162

(57) ABSTRACT

An active electrode (30) for an electrical surgical instrument (20) includes a plurality of strand-type or cord-type structures (42, 52) that are disposed next to one another as well as an electrode area. The electrode area includes surface regions (48, 58) of each of the plurality of strand-type or cord-type structures (42, 52).

### CROSS REFERENCE TO RELATED APPLICATIONS

This application is a United States National Phase Application of International Application PCT/EP2022/068701, filed Jul. 6, 2022, and claims the benefit of priority under 35 U.S.C. § 119 of German Application 10 2021 117 566.6, filed Jul. 7, 2021, the entire contents of which are incorporated herein by reference.

### TECHNICAL FIELD

The present invention relates to an electrosurgical instrument, in particular a resectoscope or a hysteroscope for urological or gynecological measures, to an active electrode for an electrosurgical instrument, and to an electrosurgical system having an electrosurgical instrument.

### BACKGROUND

Tumors are ablated using electrosurgical means especially in urology and in gynecology. In this context, there increasingly is the desire to directly vaporize the ablated tissue. As a rule, a bipolar resectoscope used to this end comprises a neutral electrode and an active electrode. The neutral electrode has a surface that is as large as possible, in order to create current densities that are as low as possible there and hence, as far as possible, no electrosurgical effect. The active electrode is significantly smaller and is also referred to as a ball electrode on account of—especially in the past—frequently having a spherical shape. A small part of the surface of the active electrode, which for example faces the inner wall of the ureter or the mucous membrane of cervix or uterus, is electrosurgically effective. A high current density arises there, and can bring about electrocauterization or a vaporization of tissue.

DE 698 36 640 T2 describes an apparatus for treating tissue using multiple electrodes. A multiplicity of elastic electrodes are pushed out of a cannula distally and curve along arcuate trajectories on the distal side of the cannula on account of spring memory.

U.S. Pat. No. 7,181,288 B1 describes a similar arrangement, albeit not for electrosurgery but for the stimulation of areas of the brain.

DE 60 2005 004 630 T2 describes an instrument for treating hemorrhoid tissue, in which a proximal arcuate electrode arrangement is slidably attached to rails and movable relative to a distal arcuate electrode arrangement.

EP 2 149 342 A1 describes a multiphase electrosurgical system having an array of electrodes.

### SUMMARY

An object of the present invention consists of developing an improved electrosurgical instrument, an improved active electrode for such an electrosurgical instrument, an improved electrosurgical system having such an electrosurgical instrument, and an improved method for producing an active electrode.

This object is achieved by features as disclosed herein.

Various embodiments are disclosed herein.

An active electrode for an electrosurgical instrument comprises a plurality of adjacently arranged strand-shaped or bead-shaped structures and an electrode area comprising surface regions of each of the plurality of strand-shaped or bead-shaped structures.

In particular, the active electrode is provided and configured for a resectoscope or a hysteroscope. The active electrode may be connected to an electrosurgical instrument in non-separable or not readily separable fashion. Alternatively, the active electrode can be a replaceable constituent part of an electrosurgical instrument or a replaceable or non-replaceable component for an electrosurgical instrument.

In particular, a strand-shaped structure has a straight or curved rod shape having a constant cross section or having a continuously or abruptly varying cross section. In particular, a bead-shaped structure is a convex structure protruding in line-shaped or strip-shaped fashion, having a constant or continuously or abruptly varying cross section. Among themselves, the plurality of strand-shaped or bead-shaped structures may be the same or similar (in the colloquial sense or in the mathematical sense).

A plurality of strand-shaped or bead-shaped structures are adjacently arranged if each of the plurality of strand-shaped or bead-shaped structures has at least one portion, for which it is true that each plane intersecting this strand-shaped or bead-shaped structure orthogonally within this portion (more precisely: orthogonally to the curve on which the area centroids of the cross sections are located) also intersects the further one or all further ones of the plurality of strand-shaped or bead-shaped structures.

The electrode area effective during the envisaged use in particular comprises only surface regions of strand-shaped or bead-shaped structures. During envisaged use, the installation of the active electrode with regards to arrangement, orientation, and fastening, and operating parameters such as current, voltage, frequency, and impedance of the radiofrequency source, and the use by medical staff meet the requirements of the legal framework regarding medical devices and the manufacturer regulations.

Concentrating the effective electrode area to the strand-shaped or bead-shaped structures allows the effective electrode area of the active electrode to be small, and hence allows the current density to be high. This can enable a particularly reliable electrosurgical effect, in particular electrocauterization or even vaporization of tissue. At the same time, this electrosurgical effect can create an effective region corresponding to the overall dimensions of the active electrode, comparable to the effective region achieved by a conventional ball electrode.

In the case of an active electrode as described herein, in each case both ends of each of the plurality of adjacently arranged strand-shaped or bead-shaped structures are in particular mechanically rigidly connected to one or more others of the plurality of strand-shaped or bead-shaped structures or to one or more other structures.

By virtue of in each case both ends of each of the plurality of adjacently arranged strand-shaped or bead-shaped structures being mechanically rigidly connected to one or more others of the plurality of strand-shaped or bead-shaped structures or to one or more other structures, the plurality of adjacently arranged strand-shaped or bead-shaped structures can form a mechanically robust component. In particular, this mechanically robust component does not have freely protruding ends of strand-shaped or bead-shaped structures that could easily be damaged or could easily cause damage, and at which moreover particularly high electric fields could occur.

Alternatively, the active electrode may comprise further strand-shaped or bead-shaped structures in addition to the plurality of adjacently arranged strand-shaped or bead-shaped structures whose both ends in each case are connected to others of the plurality of adjacently arranged strand-shaped or bead-shaped structures or to other structures. Ends of these further strand-shaped or bead-shaped structures can be arranged freely or openly, which is to say cannot be connected to others of the plurality of adjacently arranged strand-shaped or bead-shaped structures or other structures.

In the case of an active electrode as described herein, the strand-shaped or bead-shaped structures each have an arcuate embodiment in particular.

In particular, each strand-shaped or bead-shaped structure has a circular-arc-shaped embodiment or has the form of a section of an ellipse or a parabola.

An arcuate embodiment of the strand-shaped or bead-shaped structures allows the form of the effective region of the active electrode to approximate to the form of the active region of a conventional active electrode with a curved electrode surface in the form of a section of a spherical surface.

In the case of an active electrode as described herein, the strand-shaped or bead-shaped structures are arranged in particular parallel to one another or like great circles on a spherical surface.

This arrangement of the strand-shaped or bead-shaped structures can enable the aforementioned approximation of the effective region of the active electrode to that of a conventional active electrode. An arrangement similar to great circles on a spherical surface can enable a mechanical connection and mounting and an electrical contacting of ends of strand-shaped structures at two spaced-apart “poles” (intersections of the great circles) and thus enable a mechanically and electrically robust structure.

In the case of an active electrode as described herein, the adjacently arranged strand-shaped or bead-shaped structures are in particular connected in web-like fashion by further strand-shaped or bead-shaped structures running thereacross.

In the case of an active electrode as described herein, the adjacently arranged strand-shaped or bead-shaped structures are in particular mechanically rigidly connected in web-like fashion by further strand-shaped or bead-shaped structures running thereacross.

The plurality of adjacently arranged strand-shaped or bead-shaped structures and the further strand-shaped or bead-shaped structures running thereacross are in particular arranged orthogonally or substantially orthogonally to one another. The plurality of adjacently arranged strand-shaped or bead-shaped structures and the further strand-shaped or bead-shaped structures running thereacross may intersect or penetrate one another or only be in tangential contact. The plurality of adjacently arranged strand-shaped or bead-shaped structures and the further strand-shaped or bead-shaped structures running thereacross may have the same or different cross sections.

At points of intersection or points of contact, the plurality of adjacently arranged strand-shaped or bead-shaped structures on the one hand and the further strand-shaped or bead-shaped structures running thereacross on the other hand are in each case mechanically rigidly connected, for example joined, in particular. As a result, the strand-shaped or bead-shaped structures and the further strand-shaped or bead-shaped structures running thereacross form a mechanically rigid web-like structure in particular.

The further strand-shaped or bead-shaped structures running across may mechanically reinforce the active electrode and improve its mechanical robustness. Further, they may reduce the risk of the active electrode being immersed in tissue and tissue in the process remaining between the strand-shaped or bead-shaped structures, for example without being vaporized.

In the case of an active electrode as described herein, in particular a web structure or a perforated sheet structure or a planar structure is arranged between the strand-shaped or bead-shaped structures.

The web structure or the perforated sheet structure, or the planar structure largely or completely fills the area circumscribed by the strand-shaped or bead-shaped structures.

A web structure or a perforated sheet structure or a planar structure between the strand-shaped or bead-shaped structures may mechanically reinforce the active electrode and improve its mechanical robustness. Further, a web structure or a perforated sheet structure or a planar structure between the strand-shaped or bead-shaped structures may prevent the active electrode from being immersed in tissue and tissue in the process remaining between the strand-shaped or bead-shaped structures, for example without being vaporized.

In the case of an active electrode as described herein, the web structure or the perforated sheet structure or the planar structure is in particular recessed vis-à-vis the strand-shaped or bead-shaped structures.

The recessed arrangement of the web structure or the perforated sheet structure or the planar structure may enable a concentration of the electrosurgical effect on the strand-shaped or bead-shaped structures and hence on a comparatively small effective electrode area. This may allow smaller currents and a better control characteristic.

In the case of an active electrode as described herein, a strand-shaped or bead-shaped structure in particular has a circular cross section or a cross section with a circular-arc-shaped edge portion.

In particular, each individual strand-shaped or bead-shaped structure has a circular cross section or a cross section with a circular-arc-shaped edge portion. For example, a circular cross section is obtainable if a strand-shaped structure is formed from a wire with a circular cross section. A circular-arc-shaped edge portion of a cross section extends in particular over an angular range of at least 120 degrees or at least 180 degrees.

A circular cross section or a cross section with circular-arc-shaped edge portions may enable a uniform field strength, a uniform current density, and hence also a uniform electrosurgical effect.

In the case of an active electrode as described herein, the radii of the circular cross sections or the circular-arc-shaped edge portions of the cross sections are in particular in the range from 0.1 mm to 0.3 mm or in the range from 0.1 mm to 0.5 mm.

In the case of a strand-shaped structure with a circular cross section, the diameter thereof therefore is in the range from 0.2 mm to 0.6 mm, in particular in the range from 0.3 mm to 0.5 mm.

In the case of an active electrode as described herein, neighboring strand-shaped or bead-shaped structures have in particular a maximum spacing in the range from 0.1 mm to 0.5 mm or in the range from 0.1 mm to 1 mm.

If the strand-shaped or bead-shaped structures are arranged like great circles on a spherical surface, the spacings of the structures reduce toward the “poles”.

In the case of an active electrode as described herein, in particular at least one strand-shaped structure is formed by an additive method or by an injection molding method.

A plurality of, or all, strand-shaped structures can be produced in an additive method, in particular in the same additive method, which is to say simultaneously or directly in succession. Additive methods, often also referred to as 3-D printing, provide extensive freedom in terms of shaping. Laser sintering or electron beam sintering allows the additive manufacture of structures from materials with very high melting temperatures.

In the case of an active electrode as described herein, in particular at least one strand-shaped structure is formed from a wire and joined to the remaining strand-shaped or bead-shaped structures.

A plurality of, or all, strand-shaped structures and optionally also the aforementioned further strand-shaped structures arranged thereacross may be formed from wire in each case. To this end, the wire is in particular also plastically deformed, specifically bent. The structures can be connected by interlocking, for example by virtue of a wire with a smaller cross section being arranged in a drilled hole of appropriate cross section in a wire with a larger cross section. In an alternative or in addition, the structures can be joined by laser welding for example.

An active electrode as described herein in particular comprises a first electrode component comprising a strand-shaped or bead-shaped structure (52) and a cutout, in which a portion of the wire is arranged as second electrode component.

For example, the cutout is a drilled hole, into which one end of the wire is inserted, or a drilled through-hole, through which the wire protrudes. In particular, the wire forms one or more of the plurality of strand-shaped structures and a part of the effective electrode area of the active electrode. The first electrode component may comprise a plurality of strand-shaped or bead-shaped structures and/or a plurality of cutouts. For example, the first electrode component is produced by means of an additive method.

A combination of electrode components produced by means of different production methods may enable a combination of the advantages of various production methods. For example, the first electrode component produced by means of an additive method may have a spatial form that is not attainable, or only attainable with great outlay, using other methods, while the wire may allow greater mechanical robustness and, at the same time, electrical contacting over a greater distance.

An active electrode for an electrosurgical instrument comprises a web structure or a perforated sheet structure which forms an electrode area of the active electrode.

In particular, the active electrode has no structures protruding beyond the electrode area formed by the web structure or perforated sheet structure.

In particular, the electrode area is formed exclusively by the web structure or perforated sheet structure. Alternatively, the edges of the electrode area, for example, could be formed by solid structures without meshes or holes. What also applies to these solid structures is, in particular, that they do not protrude beyond the electrode area formed by the web structure or perforated sheet structure.

The effective electrode area is smaller in the case of a web structure or a perforated sheet structure than in the case of a structure of the same size that covers the whole area. As a result, stronger electric fields can be created in the case of otherwise the same or similar geometry and the same voltage. Higher current densities can be created to a higher extent as a result thereof and as a result of concentrating the current on a smaller electrode area.

The web structure or the perforated sheet structure is in particular bent (i.e., K=0 applies to the Gaussian curvature K of a smooth surface approximating the web structure or perforated sheet structure) or curved (i.e., K>0). Alternatively K<0 may apply to the Gaussian curvature K of a smooth surface approximating the web structure or perforated sheet structure.

An active electrode as described herein is formed from tungsten in particular.

Tungsten has advantageous electrical properties, especially also in relation to the ignition of a plasma. Further, according to current knowledge, tungsten is physiologically harmless.

An electrosurgical instrument comprises an active electrode as described herein.

In particular, the electrosurgical instrument is a resectoscope for urological applications or a hysteroscope for gynecological applications.

An electrosurgical system comprises a radiofrequency generator for generating an alternating RF voltage and an electrosurgical instrument as described herein, wherein the active electrode of the electrosurgical instrument and a neutral electrode are connected to the radiofrequency generator, in order to close a circuit via the body of a patient.

An electrosurgical system comprises a radiofrequency generator for generating an alternating RF voltage and an electrosurgical instrument as described herein, wherein the active electrode of the electrosurgical instrument and a neutral electrode are connected to the radiofrequency generator, in order to close a circuit via the body of a patient, wherein the radiofrequency generator and the active electrode are configured for tissue vaporization in particular.

A method for producing an active electrode for an electrosurgical instrument comprises providing an electrically conductive wire, mechanically deforming the electrically conductive wire in order to create a first electrode component with an arcuate portion, creating a second electrode component, and joining the second electrode component to the first electrode component in the region of the arcuate portion or to the proximal side of the arcuate portion, wherein both a surface region of the first electrode component and a surface region of the second electrode component of the wire each form a part of an electrode area of the active electrode.

In particular, an active electrode as described herein is producible by means of the method. An active electrode as described herein is producible in particular by means of a method as described herein.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic illustration of an electrosurgical instrument;

FIG. 2 is a schematic illustration of an electrosurgical system;

FIG. 3 is a schematic illustration of an active electrode;

FIG. 4 is a further schematic illustration of the active electrode from FIG. 3;

FIG. 5 is a schematic illustration of a further active electrode;

FIG. 6 is a further schematic illustration of the active electrode from FIG. 5;

FIG. 7 is a schematic illustration of a further active electrode;

FIG. 8 is a further schematic illustration of the active electrode from FIG. 7;

FIG. 9 is a schematic illustration of a further active electrode;

FIG. 10 is a further schematic illustration of the active electrode from FIG. 9; and

FIG. 11 is a schematic illustration of a method for producing an active electrode.

### DESCRIPTION OF PREFERRED EMBODIMENTS

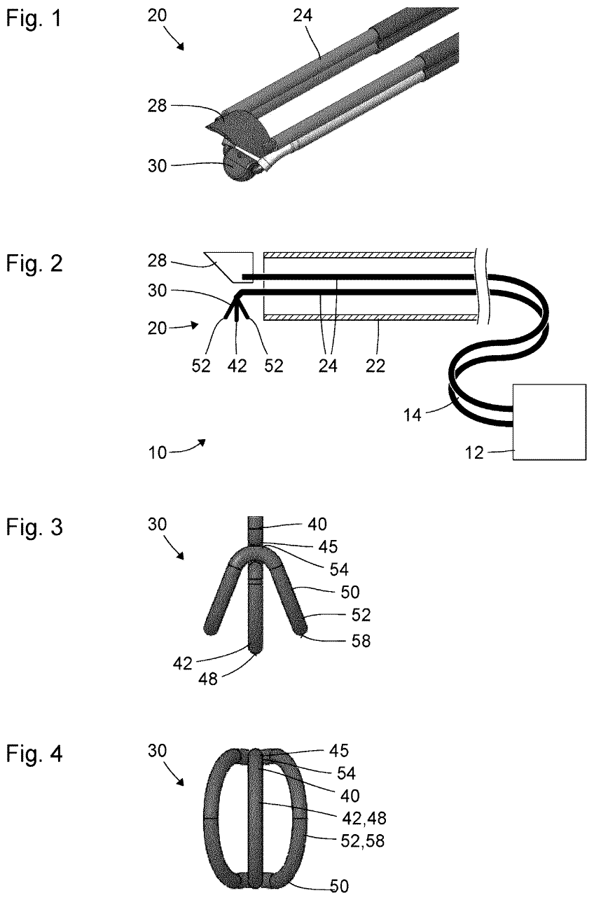

Referring to the drawings, FIG. 1 shows a schematic illustration of parts of a distal end of an electrosurgical instrument 20. In particular, the electrosurgical instrument 20 is a resectoscope or a hysteroscope for urological or gynecological applications, respectively.

The electrosurgical instrument 20 comprises electrical feed lines 24 in the form of electrically insulated wires made of electrically conductive materials. In particular, the electrical feed lines connect a plug-in connector at a proximal end (not depicted in FIG. 1) of the electrosurgical instrument 20 to a neutral electrode 28 and an active electrode 30 at the distal end depicted in FIG. 1. In particular, the electrical feed lines 24 are arranged in a shaft tube not depicted in FIG. 1. At the same time, the electrical feed lines 24 may be provided and configured for the mechanical hold and positioning of the neutral electrode 28 and the active electrode 30 at envisaged positions.

The neutral electrode 28 has a large surface region in the form of a section of a lateral surface of a circular cylinder. In the embodiment depicted in FIG. 1, the active electrode 30 has a conventional design.

FIG. 2 shows a schematic illustration of parts of an electrosurgical system 10. The electrosurgical system 10 comprises a radiofrequency generator 12 for providing an alternating RF voltage. The radiofrequency generator 12 is connected by way of a two-pole electrical line 14 to a plug-in connector at a proximal end (not depicted in FIG. 2) of an electrosurgical instrument 20. FIG. 2 only depicts a distal end region of the electrosurgical instrument 20.

In terms of some features and properties, the electrosurgical instrument 20 of the electrosurgical system 10 is similar to the electrosurgical instrument presented on the basis of FIG. 1. The electrical feed lines 24 of the electrosurgical instrument 20 are arranged in a shaft tube 22. Only the shaft tube 22 is depicted in FIG. 2 in a section along a plane containing the longitudinal axis and axis of symmetry of the shaft tube 22.

The neutral electrode 28 and the active electrode 30 are arranged distally of the distal end of the shaft tube 22 and hence outside of the shaft tube 22. The cross section of the shaft tube 22 is adapted to the envisaged use, for example for insertion into the ureter or cervix. In the depicted example, the neutral electrode 28 and the active electrode 30 are arranged such that they do not protrude beyond the outer contour of the shaft tube 22, but terminate flush with the distally straight continuation of the outer lateral surface of the shaft tube 22.

Alternatively, and deviating from the illustration in FIG. 2, the neutral electrode 28 and the active electrode 30 can be embodied and arranged such that they do not protrude beyond the inner contour of the shaft tube 22 but instead are arranged within the distally straight continuation of the inner lateral surface of the shaft tube 22. This may allow the neutral electrode 28 and the active electrode 30 to be able to be inserted through the shaft tube only after the arrangement of the latter in situ.

Unlike the active electrode of the electrosurgical instrument shown in FIG. 1, the active electrode 30 of the electrosurgical instrument shown in FIG. 2 comprises three arcuate strand-shaped structures 42, 52, which form the effective electrode area of the active electrode 30.

The radiofrequency generator 12 generates an alternating RF voltage. The frequency and the amplitude of the alternating voltage, the maximum current, the impedance of the power output of the radiofrequency generator 12, its current-voltage characteristic curve, and/or further parameters may be predetermined or adjustable. Further, the radiofrequency generator can be provided and configured to for example detect the current and its time dependence and, on the basis thereof, vary the voltage provided.

The radiofrequency generator 12 is connected to the neutral electrode 28 and the active electrode 30 by way of the two-pole electrical line 14 and the electrical feed lines 24 in the shaft tube 22. If the neutral electrode 28 for example rests against the inner wall of an ureter and the active electrode 30 likewise rests against tissue in the body of a patient or is connected thereto, for example via an electrically conductive plasma, an alternating current can flow from the radiofrequency generator 12 back to the radiofrequency generator 12 via one pole of the two-pole electrical line 14, one of the electrical feed lines 24, the active electrode 30, the body of the patient, the neutral electrode 28, the other one of the electrical feed lines 24, and the other pole of the two-pole electrical line 14.

The effective electrode area of the active electrode 30 is significantly smaller than the electrode area of the neutral electrode 28. As a consequence, the current density, the electric field arising on account of the electrical resistance, and, as a result, the power density at the active electrode 30 are significantly greater than at the neutral electrode 28. The electric power emitted by the radiofrequency generator 12 can therefore be set so that there is no physiologically effective heating of tissue and hence no electrosurgical effect at the neutral electrode 28, while at the same time tissue is significantly heated, coagulated, or even vaporized at the active electrode 30.

FIG. 3 shows a schematic illustration, enlarged in relation to FIG. 2, of the active electrode 30. The plane of the drawing in FIG. 3 corresponds to that of FIG. 2, which is to say is parallel to the longitudinal axis of the shaft of the electrosurgical instrument for which the active electrode 30 is provided.

The active electrode 30 comprises a first electrode component 40 and a second electrode component 50. In the illustrated example, both the first electrode component 40 and the second electrode component 50 are formed in each case from a tungsten wire and have the same circular cross section.

The first electrode component 40 is formed from a piece of wire bent substantially into a U-shape in a plane orthogonal to the plane of the drawing in FIG. 3. The arc of the U-shape forms an arcuate portion 42 of the first electrode component 40, and hence an arcuate strand-shaped structure of the active electrode 30 at the same time.

The second electrode component 50 has a circular topology. Two respectively arcuate portions 52 of the second electrode component 50 each form a further arcuate strand-shaped structure of the active electrode 30. Each of the two arcuate portions 52 of the second electrode component 50 is located in a plane orthogonal to the plane of the drawing in FIG. 3, wherein both planes intersect one another in a straight line. Hence, the second electrode component 50 has two strongly curved transition regions between the two arcuate portions 52.

These strongly curved transition regions between the arcuate portions 52 are joints 54 at the same time, which are mechanically connected to corresponding joints 45 on the first electrode component 40. In particular, each joint 45 on the first electrode component 40 is configured similar to an expansion bend in a district heating pipe, with two 90 degree bends, between which a 180 degree bend is arranged. The joint 54 of the second electrode component 50 is inserted into the recess arising thus. In addition to the resultant interlocking connection, the joints 45, 54 are connected by laser welding in particular.

The arcuate portion 42 of the first electrode component 40 and the arcuate portions 52 of the second electrode component 50 each represent portions of circular arcs in particular, which are arranged on a spherical surface like great circles or similar to great circles. Alternatively, the arcuate portions 42, 52 of the electrode components 40, 50 can each be formed as portions of ellipses for example.

The surface regions, oriented downward in FIG. 3, of the arcuate portions 42, 52 of the active electrode 30 face the surface to be worked on during the envisaged use and form electrode areas 48, 58, which together form the effective electrode area of the active electrode 30.

FIG. 4 shows a further schematic illustration of the active electrode 30 from FIG. 3. The plane of the drawing in FIG. 4 is orthogonal to the plane of the drawing in FIG. 3 and likewise parallel to the longitudinal axis of the shaft of the electrosurgical instrument for which the active electrode 30 is provided.

The surface regions, depicted in FIG. 4 and facing the observer, of the arcuate portions 42, 52 are largely identical to the electrode areas 48, 58 of the arcuate portions 42, 52 which form the active electrode area of the work electrode 30.

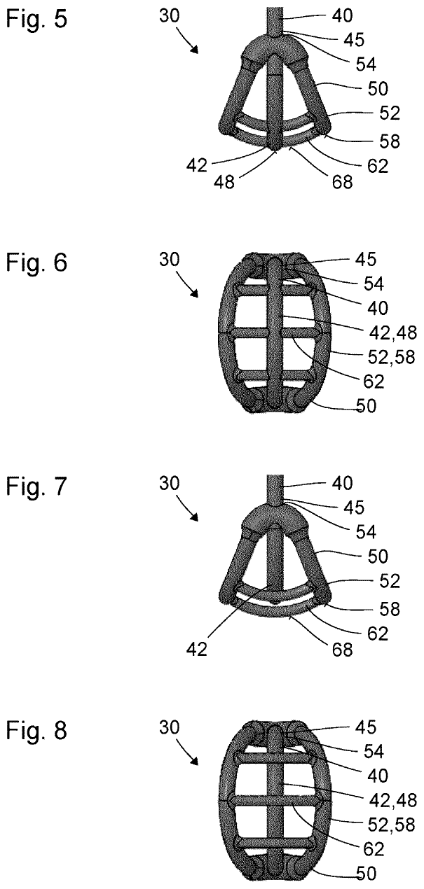

FIG. 5 shows a schematic illustration of a further embodiment of an active electrode 30, which is similar in certain features, properties, and functions to the embodiment presented on the basis of FIGS. 3 and 4. The type of illustration in FIG. 5, in particular the plane of the drawing in FIG. 5, corresponds to that of FIG. 3. In particular, features, properties, and functions by way of which the active electrode 30 shown in FIG. 5 differs from the active electrode presented on the basis of FIGS. 3 and 4 are described below.

The active electrode 30 shown in FIG. 5 differs from the active electrodes presented on the basis of FIGS. 3 and 4 in particular by way of further strand-shaped structures 62, which are arranged across the arcuate portions 42, 52 and mechanically connect these. In the illustrated example, the cross sections of the further strand-shaped structures 62 are smaller than the cross sections of the arcuate portions 42, 52. The further strand-shaped structures 62 may likewise be manufactured from wire. Their ends may be arranged in cutouts in the arcuate portions 42, 52 and/or joined thereto, for example by laser welding.

The active electrode 30 shown in FIG. 5 also differs from the active electrode presented on the basis of FIGS. 3 and 4 by way of a different configurations of the joints 45, 54 of the electrode components 40, 50. The joint 45 on the first electrode component 40 has a straight embodiment. The joint 54 on the second electrode component 50 has an enlarged cross section and a drilled through hole, in which the joint 45 of the first electrode component is arranged. The electrode components 40, 50 can be connected by laser welding at the edge of the drilled through hole.

FIG. 6 shows a further schematic illustration of the active electrode 30 from FIG. 5. The type of illustration in FIG. 6, in particular the plane of the drawing in FIG. 6, corresponds to that of FIG. 4. The plane of the drawing in FIG. 6 thus is orthogonal to the plane of the drawing in FIG. 5 and likewise parallel to the longitudinal axis of the shaft of the electrosurgical instrument for which the active electrode 30 is provided.

In the depicted example, the arcuate portions 42, 52 and the further strand-shaped structures enclose approximately square regions.

The two differences between the embodiments of FIGS. 3 and 4 and FIGS. 5 and 6 are independent of one another. The embodiment presented on the basis of FIGS. 3 and 4 may also comprise further strand-shaped structures 62 across the arcuate portions 43, 53. Alternatively, the joints 45, 54 in the embodiment presented on the basis of FIGS. 3 and 4 may be configured like in the embodiment presented on the basis of FIGS. 5 and 6.

FIG. 7 shows a schematic illustration of a further embodiment of an active electrode 30, which is similar in certain features, properties, and functions to the embodiments presented on the basis of FIGS. 3 to 6. The type of illustration in FIG. 7, in particular the plane of the drawing in FIG. 7, corresponds to that of FIGS. 3 and 5. In particular, features, properties, and functions by way of which the active electrode 30 shown in FIG. 7 differs from the active electrodes presented on the basis of FIGS. 3 to 6 are described below.

The active electrode 30 shown in FIG. 7 differs from the active electrode presented on the basis of FIGS. 5 and 6 in particular in that the further strand-shaped structures 62 do not intersect the arcuate portion 42 and, in the depicted example, do not come into contact therewith either. This can simplify manufacturing if initially the first electrode component is guided through the drilled through holes in the joints 54 in the second electrode component 50—in relation to FIG. 7: in a movement from top to bottom—and subsequently the further strand-shaped structures 62 are positioned and their ends are mechanically connected to the arcuate portions 52.

Deviating from the illustration in FIG. 7, the further strand-shaped structures 62 may be joined to the arcuate portion 42, for example by laser welding.

Deviating from the depiction in FIG. 7, the joints 45, 54 of the active electrode 30 shown in FIG. 7 can for example be embodied as presented on the basis of FIGS. 3 and 4.

FIG. 8 shows a further schematic illustration of the active electrode 30 from FIG. 7. The type of illustration in FIG. 8, in particular the plane of the drawing in FIG. 8, corresponds to that of FIGS. 4 and 6. The plane of the drawing in FIG. 8 thus is orthogonal to the plane of the drawing in FIG. 7 and likewise parallel to the longitudinal axis of the shaft of the electrosurgical instrument for which the active electrode 30 is provided.

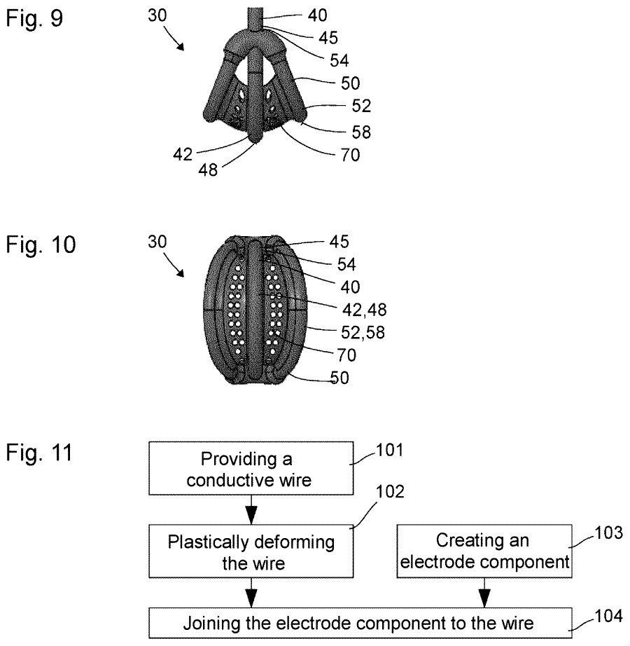

FIG. 9 shows a schematic illustration of a further embodiment of an active electrode 30, which is similar in certain features, properties, and functions to the embodiment presented on the basis of FIGS. 3 and 4 and in particular the embodiments presented on the basis of FIGS. 5 to 8. The type of illustration in FIG. 9, in particular the plane of the drawing in FIG. 9, corresponds to that of FIGS. 3, 5 and 7. In particular, features, properties, and functions by way of which the active electrode 30 shown in FIG. 9 differs from the active electrodes presented on the basis of FIGS. 3 to 8 are described below.

The active electrode 30 shown in FIG. 9 differs from the active electrodes presented on the basis of FIGS. 3 to 8 in particular in that perforated sheet structures 70 are provided between the arcuate portions 42, 52. The holes or drilled through holes in the perforated sheet structure 70 can simplify aspiration of vapors arising during electrocauterization.

Apart from the holes in the perforated sheet structure 70, the active electrode 30 has a configured with a curved area with three bead-shaped structures, which are formed by the arcuate portions 42, 52. The bead-shaped structures formed by the arcuate portions 42, 52 each have a substantially more pronounced convex configuration with significantly smaller radii of curvature in comparison with the perforated sheet structures 70. Hence, significantly stronger electric fields and correspondingly higher current densities and substantially higher power densities are present in the regions of the bead-shaped structures. Thus, the electrosurgical effect is largely concentrated on these bead-shaped structures formed by the arcuate portions 42, 52. Accordingly, the effective electrode area 48, 58 during the envisaged use is substantially formed by the downwardly oriented—in relation to FIG. 9—surface regions of the bead-shaped structures formed by the arcuate portions 42, 52.

FIG. 10 shows a further schematic illustration of the active electrode 30 from FIG. 9. The type of illustration in FIG. 10, in particular the plane of the drawing in FIG. 10, corresponds to that of FIGS. 4, 6 and 8. The plane of the drawing in FIG. 10 thus is orthogonal to the plane of the drawing in FIG. 9 and likewise parallel to the longitudinal axis of the shaft of the electrosurgical instrument for which the active electrode 30 is provided.

Both the first electrode components 40 and the second electrode components 50 can be formed from wire in each of the active electrodes 30 presented on the basis of FIGS. 3 to 10 and the further strand-shaped structures 60 can also be formed from wire in the active electrodes presented on the basis of FIGS. 5 to 8. In the active electrode 30 presented on the basis of FIGS. 9 and 10, the perforated sheet structures 70 can be manufactured from sheets. The connection of the further strand-shaped structures 60 or the perforated sheet structures 70 to the arcuate portions 42, 52 can optionally be implemented by inserting ends of the further strand-shaped structures 60 or edges of the perforated sheet structures 70 into corresponding cutouts in the arcuate portions 42, 52 and implemented in particular by integral bonding, for example by way of laser welding.

Alternatively, the respective entire active electrode 30 can be formed by an additive method, for example by laser sintering or electron beam sintering.

Alternatively, each of the active electrodes 30 presented on the basis of FIGS. 3 to 10 can be created by a hybrid method, in which only a part of the active electrode 30 is created by an additive method and another part is created in a different way. For example, the first electrode component 40 can be formed in each case by bending a wire, whereas the second electrode component—optionally already comprising the further strand-shaped structures 62 or the perforated sheet structures 70—is produced by means of an additive method. The two parts can subsequently be connected mechanically, for example by laser welding, and optionally by an interlocking connection as well, in particular by guiding the first electrode component 40 through drilled through holes in the second electrode component 50 or by inserting the first electrode component 40 into cutouts in the second electrode component 50, or vice versa.

FIG. 11 shows a schematic flowchart of a method for producing an active electrode for an electrosurgical instrument, in particular an active electrode having features, properties, and functions of the active electrodes presented on the basis of FIGS. 3 to 10. However, the method can also be used to produce active electrodes with deviating features, properties, and functions. The subsequent use of reference signs from FIGS. 3 to 10 is therefore purely by way of example.

A wire made of an electrically conductive material, for example tungsten, is provided in a first step 101. The wire is plastically deformed in a second step 102 in order to form an—in particular U-shaped—first electrode component 40.

A second electrode component 50 is created in a third step 103, for example by means of an additive method. The third step can be carried out before or after the first step 101 and the second step 102, or partly or completely at the same time thereto.

In a fourth step, which is carried out after the first step 101 and the second step 102 and after the third step 103, the first electrode component 40 and the second electrode component 50 are joined, for example by laser welding, optionally by an interlocking connection as well.

While specific embodiments of the invention have been shown and described in detail to illustrate the application of the principles of the invention, it will be understood that the invention may be embodied otherwise without departing from such principles.

### REFERENCE NUMBERS

10 Electrosurgical system
12 Radiofrequency generator of the electrosurgical system 10
14 Electrical line for connecting the electrosurgical instruments 20 to the radiofrequency generator 12
20 Electrosurgical instrument of the electrosurgical system 10
22 Shaft tube of the electrosurgical instrument 20
24 Electrical feed lines of the electrosurgical instrument 20
28 Neutral electrode of the electrosurgical instrument 20
30 Active electrode of the electrosurgical instrument 20
40 Electrically conductive wire as first electrode component of the active electrode 30
42 Arcuate portion of the first electrode component 40
45 Joint of the first electrode component 40, joined to the joint 54 of the second electrode component 50
48 Electrode area on the arcuate portion of the first electrode component 40
50 Second electrode component of the active electrode 30
52 Arcuate portion of the second electrode component 50
54 Joint of the second electrode component 50, joined to the joint 45 of the first electrode component 40
58 Electrode area on the arcuate portion of the second electrode component 50
62 Further strand-shaped or bead-shaped structure of the active electrode 30, across the arcuate portions 42, 52 of the electrode components 40, 50
68 Electrode area on the further strand-shaped or bead-shaped structure 60
70 Perforated sheet structure between the strand-shaped or bead-shaped structures 42, 52
101 First step (providing an electrically conductive wire)
102 Second step (mechanically deforming the electrically conductive wire)
103 Third step (creating an electrode component)
104 Fourth step (joining the electrode component to the wire)

1. An active electrode for an electrosurgical instrument, the active electrode comprising:
a plurality of adjacently arranged strand-shaped or bead-shaped structures; and
an electrode area comprising surface regions of each of the plurality of strand-shaped or bead-shaped structures.

2. The active electrode as claimed in claim 1, wherein the plurality of strand-shaped or bead-shaped structures each include ends and each of both ends of each of the plurality of adjacently arranged strand-shaped or bead-shaped structures are mechanically rigidly connected to one or more ends of other of the plurality of strand-shaped or bead-shaped structures.

3. The active electrode as claimed in claim 1, wherein the strand-shaped or bead-shaped structures each have an arcuate form.

4. The active electrode as claimed in claim 1, wherein the strand-shaped or bead-shaped structures are arranged parallel to one another or as a portion of circles on a spherical surface.

5. The active electrode as claimed in claim 1, wherein the adjacently arranged strand-shaped or bead-shaped structures are mechanically rigidly connected in a web form by further strand-shaped or bead-shaped structures running thereacross.

6. The active electrode as claimed in claim 1, wherein a web structure or a perforated sheet structure or a planar structure is arranged between the strand-shaped or bead-shaped structures.

7. The active electrode as claimed in claim 1, wherein at least one of the strand-shaped or bead-shaped structures has a circular cross section or a cross section with a circular-arc-shaped edge portion.

8. The active electrode as claimed in claim 7, wherein radii of the circular cross sections or the circular-arc-shaped edge portions of the cross sections are in the range from 0.1 mm to 0.5 mm.

9. The active electrode as claimed in claim 1, wherein neighboring strand-shaped or bead-shaped structures have a maximum spacing in the range from 0.1 mm to 1 mm.

10. The active electrode as claimed in claim 1, wherein at least one strand-shaped structure is formed by an additive method or by an injection molding method.

11. The active electrode as claimed in claim 1, wherein at least one strand-shaped structure is formed from a wire and joined to remaining strand-shaped or bead-shaped structures.

12. The active electrode as claimed in claim 11, further comprising: a first electrode component comprising at least one of the strand-shaped or bead-shaped structures and at least one cutout, in which a portion of the wire is arranged as a second electrode component.

13. An electrosurgical instrument comprising an active electrode, according to claim 1.

14. An electrosurgical system comprising:
a radiofrequency generator for generating an alternating RF voltage;
an electrosurgical instrument comprising an active electrode, the active electrode comprising: a plurality of adjacently arranged strand-shaped or bead-shaped structures; and an electrode area comprising surface regions of each of the plurality of strand-shaped or bead-shaped structures; and
a neutral electrode,
wherein the active electrode of the electrosurgical instrument and the neutral electrode are connected to the radiofrequency generator, in order to close a circuit via the body of a patient,
wherein the radio frequency generator and the active electrode are configured for tissue vaporization.

15. A method for producing an active electrode for an electrosurgical instrument, the method comprising the steps of:
providing an electrically conductive wire;
mechanically deforming the electrically conductive wire in order to create a first electrode component with an arcuate portion;
creating a second electrode component;
joining the second electrode component to the first electrode component in the region of the arcuate portion or to the proximal side of the arcuate portion,
wherein both a surface region of the first electrode component and a surface region of the second electrode component of the wire each form a part of an electrode area of the active electrode.

16. A method according to claim 15, wherein the active electrode comprises a plurality of adjacently arranged strand-shaped or bead-shaped structures with an electrode area comprising surface regions of each of the plurality of strand-shaped or bead-shaped structures, wherein the first electrode component comprises at least one of the strand-shaped or bead-shaped structures and at least one cutout, in which a portion of the wire is arranged as the second electrode component.

17. A method according to claim 16, wherein the plurality of strand-shaped or bead-shaped structures each include ends and each of both ends of each of the plurality of adjacently arranged strand-shaped or bead-shaped structures are mechanically rigidly connected to one or more ends of other of the plurality of strand-shaped or bead-shaped structures.

18. A method according to claim 16, wherein a web structure or a perforated sheet structure or a planar structure is arranged between the strand-shaped or bead-shaped structures.

19. A method according to claim 16, wherein at least one of the strand-shaped or bead-shaped structures has a circular cross section or a cross section with a circular-arc-shaped edge portion and radii of the circular cross sections or the circular-arc-shaped edge portions of the cross sections are in the range from 0.1 mm to 0.5 mm.

20. A method according to claim 16, wherein neighboring strand-shaped or bead-shaped structures have a maximum spacing in the range from 0.1 mm to 1 mm.
